(19) United States
(12) Patent Application Publication
(10) Pub. No.: US 2025/0161658 A1
(43) Pub. Date: May 22, 2025

(54) DEVICE FOR ASSISTING OR SUBSTITUTING THE HEART

(71) Applicant: BYPA MEDICAL SOLUTIONS, Dijon (FR)

(72) Inventor: Bertrand DE BROSSES, Dijon (FR)

(73) Assignee: BYPA MEDICAL SOLUTIONS, Dijon (FR)

(21) Appl. No.: 18/725,978

(22) PCT Filed: Dec. 15, 2022
(86) PCT No.: PCT/EP2022/086065

§ 371 Date: Feb. 7, 2025

(30) Foreign Application Priority Data
Dec. 31, 2021 (FR) 2114714

(51) Int. Cl. A61M 60/531; A61M 60/126; A61M 60/258; A61M 60/268; A61M 60/38; A61M 60/441

(52) U.S. Cl. CPC A61M 60/531; A61M 60/126; A61M 60/258; A61M 60/268; A61M 60/38; A61M 60/441

(57) ABSTRACT

The invention relates to a temporary circulatory assistance or support device for the heart (13) of a patient (12), a first pressure sensor (50) being disposed in the admission portion (19) and this pressure sensor (50) having a measurement time constant of less than 20 milliseconds so that the control means (40) modulate or stop the pumping of the extracted blood when the pressure detected by the sensor (50) attains a threshold pressure value and/or the pressure increases/decreases above or below a threshold pressure acceleration/deceleration slope.

### TECHNICAL FIELD

The present invention relates to a device for assisting or supporting the heart and advantageously the lungs (oxygenator).

The invention also relates to a device for assisting or supporting the heart including or using such an admission or ejection cannula. The term assistance is used when some of the blood reaching the heart is extracted whereas the term support is used when all, or almost all (>90%), of the blood reaching the heart is extracted by the device for supporting the heart.

It relates to the technical field of devices used for extracorporeal circulatory assistance/support which maintain haemodynamics compatible with life, in the context of life-threatening cardiac muscle failure, following acute or chronic heart failure (coronary insufficiency and/or cardiovascular disease, or other associated condition).

### PRIOR ART

Severe heart failure, or the inability of a person's heart to pump enough blood to meet their body's needs, is the cause of very poor quality of life, very high medical treatment costs and mortality for hundreds of thousands of patients each year. Many supports have been devised: pharmacological, biological and implanted device interventions, to treat this disease (failure), many of which are patented, but, despite these endeavours, heart failure remains a major public health problem.

The cardiovascular system is a pressurised closed hydraulic circuit, lined internally by endothelial cells. The endothelium (four kg per seventy kg) is continually subjected to tangential shear stress forces which are essential for maintaining endothelial function comprising vascular tone by nitric oxide synthesis (NOS), blood clotting, inflammatory response, atherosclerosis, angiogenesis and apoptosis.

Extracorporeal circulation (abbreviated as ECC) is a medical device used for replacing the heart and lungs in operating theatres, cardiac catheterisation laboratories or intensive care units, for paediatric or adult patients.

As a source of energy, a console or central ECC unit includes a mathematical model designed based on the laws of physics governing the movement of a fluid in a closed circuit.

This circuit is concretely composed of a pump, a heat exchanger, a flow meter, a blood gas and electrolyte analyser, a pressure recorder as well as biocompatible materials such as tubes, arterial and venous cannulas, the venous reservoir, oxygenator, arterial filter. Usually, a centrifugal or peristaltic pump is used as arterial head pump and four other peristaltic pumps are used for cardiotomy suction, cardiac chamber circulation, cardioplegia administration and a backup pump.

ECC is required to maintain organ perfusion and metabolic functions, oxygen transport during surgical cardioplegia or to assist the cardiac muscle during surgery. If failure persists, ECMO (“Extracorporeal Membrane Oxygenation”) or ECLS (“Extracorporeal Life Support”) is indicated. These systems are simpler than standard ECC and are transportable, the time of use being several days unlike conventional ECC. Indeed, unlike conventional ECC, ECMO and ECLS are maintained until the patient's cardiopulmonary recovery or as a pre-transplant relay.

The function of the heart is that of distributing blood in the body to transport the oxygen and nutrients required for the different organs to function. It is divided into two parts, the right heart which receives venous blood (oxygen-depleted) via the venae cavae and which sends it via the pulmonary artery to the respiratory system (where it is recharged with oxygen), and the left heart which receives oxygenated blood via the pulmonary veins to eject it via the aorta to the various organs.

Contractions of the cardiac muscle (pump) make it possible to generate a blood flow in the body. They allow the blood flow to continually serve the physiological needs of the body and of each organ (for example, they increase in heart rate and output with physical exertion).

The coronary arteries continuously supply blood to the cardiac muscle and ensure its oxygenation and its energy supply allowing its pumping function.

When these arteries constrict (induced by risk factors such as hypercholesterolaemia, hypertension, smoking, diabetes, heredity and others), the cardiac muscle is not supplied with sufficient blood and therefore not sufficiently oxygenated and it may become necrotic. The major consequence of this coronary insufficiency is essentially characterised by the inability of the cardiac muscle to generate a blood flow adapted to the body's vital needs (ischemic heart failure).

In the presence of a failure of the cardiac muscle (in the case of myocardial infarction for example), it is necessary to compensate for the pumping function of said cardiac muscle with a mechanical circulatory assistance system.

Among the circulatory assistance systems currently used, extracorporeal circulation devices are known, making it possible to short-circuit the defective heart using a controlled pump placed outside the body, which receives blood at the venae cavae and injects it at the (thoracic) aorta, to mechanically generate a continuous blood flow compatible with the body's vital needs.

Although these systems have shown their efficacy in their conventional circulatory assistance functions, they are not satisfactory:
the components of these assistance systems are sterile, pre-assembled and preconditioned. On account of this type of assembly, the possible patient and/or condition modularity remains limited;
the set-up of these devices is complex. It requires a complicated surgical procedure, in an operating theatre, surrounded by specialised human resources only found in major hospitals having an advanced technical platform;
the floor space requirement is very substantial;
the purchase price is very high;
the control of the blood flow generated does not account for the patient's physiology;
the possibility of mechanical stress on the formed elements of the blood (for example, red blood cells) suggests a non-negligible risk of haemolysis with very strong anti-coagulation;
given the high suction flow rate of the patient's blood, the risk of collapse of the patient's vena cava is substantial, or very substantial, which must be avoided in ECC/ECMO/ECLS.

The present application intends firstly to solve the latter problem associated with extracted blood haemolysis, given that in the device according to the invention, a reservoir is used and the blood flow rate is substantial, or very substantial, at least 2 to 3 or even 5 litres per minute.

Document FR 2872707 may be considered to represent the prior art that the present invention seeks to improve.

The invention aims to remedy this situation.

In particular, an essential objective of the invention is that of providing ECC/ECMO/ECLS preventing any risk of collapse at the admission or extraction of a patient's blood.

It also has the aim of allowing modulable set-up according to the failure of the blood flow generating function, an oxygenation system not being necessarily integrated in the device according to the invention.

The aim of the invention is also that of allowing the use of the device according to the invention without needing a complicated surgical procedure in an operating theatre. In this way, the device according to the invention may be used in an operating theatre, in a coronary and/or vascular angioplasty lab or an intensive care and/or resuscitation unit.

The aim of the invention is also that of enabling better performances and increased precision of pumping in the case of circulatory assistance.

The aim of the invention is also that of facilitating the adaptation of the system according to the invention to the different types of conditions encountered and to the patient's clinical status to preserve haemodynamics compatible with said patient's life.

The aim of the invention is also that of minimising the mechanical stress on the formed elements of the blood.

An additional objective is that of providing a simpler technical solution, easy to manage for the operator(s).

The aim of the invention is also that of providing a circulatory assistance device of simple design, that is mobile, easy to implement, use, thus helping reduce the associated costs significantly.

The aim of the invention is finally that of providing a circulatory assistance device of simple design, that is mobile, easy to implement, use, thus helping reduce the associated costs significantly.

### DISCLOSURE OF THE INVENTION

It was thus observed by the applicant, after various experiments and operations, that it is particularly advantageous to manage the suction of the patient's blood as finely as possible, i.e. be able to modulate or modify the suction of the blood extracted at the vena cava in order to prevent any risk of collapse. The applicant has defined a particularly simple way to solve this issue.

The solution provided by the invention is a temporary circulatory assistance and support device for a patient's heart, the device including a derivation circuit of the blood reaching the heart to move it to the output of the patient's heart, the derivation circuit using tubes interconnecting the following elements of said device:
at the admission portion of the derivation circuit, an admission cannula intended to be introduced into a vena cava of the patient to extract blood from the venous system,
at the ejection portion of the derivation circuit, an ejection cannula intended to be introduced into a patient's aorta or pulmonary artery to inject blood into the patient's arterial system,
a pumping system including a reservoir with variable internal volume, located between the admission portion and the ejection portion, for the admission of the extracted blood followed by the ejection of this blood, said pumping system being controlled by control means such that the pumping of the blood, from its admission to its ejection, accounts in real time for the patient's physiological needs and the haemodynamic status of said patient, the pumping system including at least a first pressure sensor, connected to the control means, disposed in the blood circulation circuit.

The device is remarkable in that the first pressure sensor is disposed in the admission portion and in that this pressure sensor has a measurement time constant of less than 20 milliseconds so that the control means modulate or stop the pumping of the extracted blood when the pressure increases/decreases above or below a threshold pressure acceleration/deceleration slope.

The expression “admission portion” means the sector of the blood derivation circuit located between the distal end of the admission cannula, i.e. the end of the cannula closest to the heart and the reservoir wherein the extracted blood is intended to be moved/conveyed.

Thanks to the device according to the invention, any risk of collapse at the vena cava is prevented, thanks to the two threshold values (absolute and/or acceleration slope) and continuous and rapid detection (high frequency) of the pressure in the admission portion.

Advantageously, said control means are capable of imposing a greater force 500 N (Newton) to accelerate or slow down a blood column slowly.

Further advantageous features of the device according to invention are listed below. Each of these features may be considered alone or in combination with the remarkable features defined above. Each of these features contributes, where applicable, to solving specific technical problems defined earlier in the description and in which the remarkable features defined above are not necessarily involved. The latter may, where applicable, be the subject of one or more divisional patents:

According to another possibility offered by the invention, the control means also modulate or stop the pumping of the extracted blood when the pressure detected by the sensor attains a threshold pressure value.

Preferably, the pressure sensor has a measurement time constant of less than 10 milliseconds.

Advantageously, the first pressure sensor is located in or on the admission cannula, at a distance of at most 15 centimetres from the proximal end of said cannula, or in the derivation circuit between the admission cannula and the pumping system.

The expression “proximal portion” means the part opposite the distal part on the cannula, i.e. the part extending from the end of the main body of the cannula located outside the patient's body or introduced slightly therein, relatively near the reservoir. On the other hand, the “distal portion” relating to the cannula consists of the part of the main body extending from the end near the heart, once the cannula has been inserted or conveyed into the patient's body, in other words the part which includes the suction means.

Very advantageously, the pumping system comprises a second pressure sensor located in the reservoir such that, thanks to the pressure readings of the first and the second sensor, the control means determine the viscosity of the extracted blood, this viscosity value being integrated or considered in said threshold pressure value and/or said threshold pressure acceleration/deceleration slope.

Indeed, using the pressure information from the first sensor and the pressure information from the second sensor, it is possible to calculate, according to a method known to a person skilled in the art, the viscosity of the blood at the vena cava, i.e. at the blood extraction position or location thanks to dynamic load loss measurements.

This second sensor optionally serves to confirm the pressure measurement of the first sensor, in particular if the latter is defective or returns obviously incorrect information. In this case of a malfunction of the first pressure sensor, then the second pressure sensor located in the reservoir will fulfil the function of the first pressure sensor.

Advantageously, the pumping system comprises a third pressure sensor located in the ejection portion.

This third measurement of the blood pressure makes it possible to refine the measurement of the viscosity of the patient's blood, this viscosity being used to decrease or increase slightly the two threshold values (absolute and acceleration slope).

It should be noted here that determining the blood viscosity is not essential for the implementation of the device according to the invention, but this measurement of the blood viscosity is advantageous for defining these two thresholds more precisely, in particular the extraction of blood in sufficient quantity from the vena cava at low pressure.

Advantageously, the pumping system also includes a blood oxygenation system, preferably this blood oxygenation system enabling blood oxygenation at the ejection portion.

Advantageously, the distal end of the admission cannula is located at a distance of at most 5 centimetres from the right atrium of the heart.

It should be noted that the cannulas according to the invention, i.e. the main body, have a length of around 70 centimetres (cm), in other words, a length between 60 cm and 80, preferably between 65 cm and 75 cm.

Advantageously, the reservoir has a pear shape with a base of wide diameter d₁ reducing in its height to reach a top of small diameter d₂ such that d₁ is at least equal to seven times d₂, i.e. d₁>7d₂, and in that the admission portion enters the reservoir, via an entry conduit, oriented obliquely according to an angle between 10° and 60° relative to the surface of the reservoir and upward—i.e. toward said top—according to an angle between 10° and 50°.

Advantageously, the reservoir is disposed vertically, the base extending along a horizontal plane, and the exit conduit being located symmetrically, along a perpendicular and vertical plane relative to the extension plane of the base, to the top of the reservoir.

Preferably, the internal volume of the reservoir is between 80 cm3 (cubic centimetres) and 120 cm3, preferably between 95 cm3 and 105 cm3. The reservoir advantageously has a volume of 100 cm3.

Advantageously, the ejection portion enters the reservoir, via an exit conduit, via the top such that the exit conduit has the diameter d2 as its diameter. According to a different interpretation, the ejection portion comes from the reservoir which forms the exit conduit.

Thanks to this feature, combined with that of the reservoir and the entry conduit, any bubbles generated during the entry of the blood into the reservoir or during its suction will be led naturally to escape via the ejection conduit and removed in or via a specific extraction circuit.

Advantageously, the reservoir comprises at least two tabs intended to engage on a mount for attaching the reservoir.

Advantageously, an elastomer membrane is attached under the base of the reservoir, said membrane having a movable top face forming one of the surfaces of the reservoir with a variable internal volume, top face extending, in an initial state, along the plane of the base of the reservoir.

Advantageously, said top face of the membrane of movably mounted under the action of a cylinder, each step of the cylinder corresponding to an internal volume of the reservoir.

According to a preferred execution mode, the diameter d1 at the base of the reservoir is between 8 and 12 centimetres (cm), preferably between 9 cm and 11 cm, whereas at the top, the diameter d2 is between 0.9 and 1.3 cm, preferably between 1 m and 1.2 cm.

It is understood with this example that d₁ may be equal to more than ten times d₂, i.e. d₁>10 d₂ or equal to more than eleven or 12 times d₂ (d₁>11 d₂ or d₁>12 d₂).

Advantageously, the membrane is a movable inner face under the action of an actuation means so as to increase or reduce the internal volume of the reservoir, the membrane forming one of the surfaces of the reservoir with a variable internal volume, said surface extending, in an initial state, along the plane of the base of the reservoir.

Preferably, the actuation means of the membrane is a cylinder (electric), each step of the cylinder corresponding to an internal volume of the reservoir. The cylinder acts upon the membrane via a central insert. The cylinder acts upon the membrane via a central insert, embedded in the elastomer.

This central insert ensures the transmission of the movement set by the cylinder and sets the deformation of the membrane via an optimal and deterministic profile. The cylinder is advantageously an electric cylinder allowing both a substantial power/force availability with a reactivity of the order of one hundredth of a second, which cannot be achieved with hydraulic or pneumatic cylinders at the present time. Moreover, an electric system does not require substantial maintenance.

Preferably, the deformable membrane consists of an elastomer, preferably said membrane consists of a silicone.

The present invention also relates to an admission cannula for a device for temporarily assisting or supporting a patient's heart, the admission cannula being intended to be introduced at least partially into a vena cava of the patient to a final position for extracting blood from the venous system thanks to a pumping system to which said admission cannula is connected, the cannula comprising:
a flexible long main body for blood suction, and
a lateral duct in particular for introducing a guiding mandrel intended to allow the admission cannula to be moved to its final position for blood suction,
   the main body and the lateral duct being attached to each other,
   the admission cannula is characterised in that:
the main body is at least hollow on its distal portion wherein said body includes at least one blood suction orifice, the distal portion having a diameter dr when said distal position is not filled, and
the lateral duct is moved to an initial position against the main body thus forming a first retracted position, and
said retracted position is held by a temporary wrapper housing said main body and said duct up to the final position of the cannula,
   wherein the temporary wrapper, once removed, allows the main body and the lateral duct to occupy a second developed position wherein the lateral duct is now at a distance from the main body and the distal portion of said main body is filled with a fluid such that the distal portion has a diameter dd.

Advantageously, the temporary wrapper includes at least one preformed tear line for tearing said wrapper and then removing it.

Advantageously, the diameter dd is at least equal to twice the diameter dr (dd≥2dr), preferably is equal to at least three times the diameter of (dd≥3dr).

Preferably, the main body is hollow along its entire length such that said main body has a diameter dr along its entire length when said body is not filled with fluid.

Advantageously, the lateral duct is attached to the main body in the developed position. Thus, the lateral duct is always attached to the main body, whether in its retracted position or in its developed position.

Advantageously, the main body comprises a plurality of blood suction orifices, an end orifice and a plurality of lateral orifices disposed on the circumference of the main body.

Advantageously, a rod for perforating the septum, said rod being introduced into the lateral duct when this duct is in its developed position.

Thanks to the lateral duct of the cannula, a rod for perforating the septum may be introduced. At the present time, with the cannulas of the prior art, during ECMO (“Extracorporeal membrane oxygenation”), once the left heart is not emptied due to absence of systole, clinicians are obliged to introduce a cannula into the left atrium to drain it (left drainage to prevent the risk of haemorrhagic pulmonary oedema and patient death). Thanks to this perforation rod and the lateral duct, the left drainage blood will be reinjected (recovered) in the blood derivation circuit of the device for assisting and supporting the heart.

Advantageously, the main body is made of plastic or of elastomer.

Advantageously, the fluid filling the distal portion of the main body or the main body consists of a liquid, preferably consists of water.

### BRIEF DESCRIPTION OF THE FIGURES

Further advantages and features of the invention will emerge more clearly on reading the description of a preferred embodiment hereinafter, with reference to the appended drawings, made by way of indicative and non-limiting examples and wherein:

FIG. 1 is a schematic view of the temporary circulatory assistance or support device for a patient's heart according to the invention.

FIG. 2 is another schematic view illustrating in particular the admission and ejection conduits of the temporary circulatory assistance and support device for a heart.

FIG. 3 is a representation of an embodiment of the reservoir of the temporary circulatory assistance or support device for a patient's heart according to the invention.

FIG. 4 is a bottom view of the reservoir seen in FIG. 3.

FIG. 5 is a top view of the reservoir seen in FIG. 3.

FIG. 6 is a representation of the reservoir of FIG. 3 connected or disposed in a holding mount.

FIG. 7 is a representation of an embodiment of the different constituent elements of an admission cannula of the temporary circulatory assistance or support device for a patient's heart according to the invention.

FIG. 8 is a view illustrating the admission cannula in its temporary wrapper as well as an indication of its release for the deployment of the admission cannula.

### DESCRIPTION OF THE EMBODIMENTS

The present invention will be described hereinafter in relation to a temporary cardiac circulatory assistance device of the type using a linear-displacement cylinder 10 to draw in and eject blood in a reservoir 11. Nevertheless, the present invention is not limited to this type of circulatory assistance devices and may be used with other types of circulatory assistance devices such as devices using rotary pumps (i.e. axial, centrifugal, mixed flow), well-known to those skilled in the art.

The device according to the invention is intended to be used in a life-threatening degraded haemodynamic context in the patient 12 (tissue perfusion pressure—PF—less than 50 mm Hg). It makes it possible to assist or support the heart 13 of a patient 12.

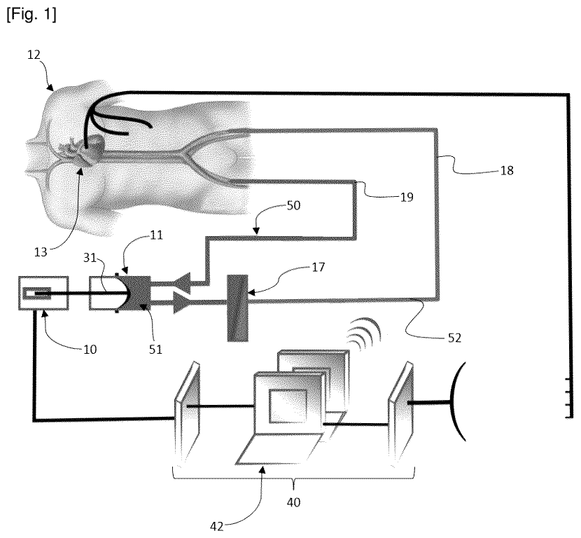

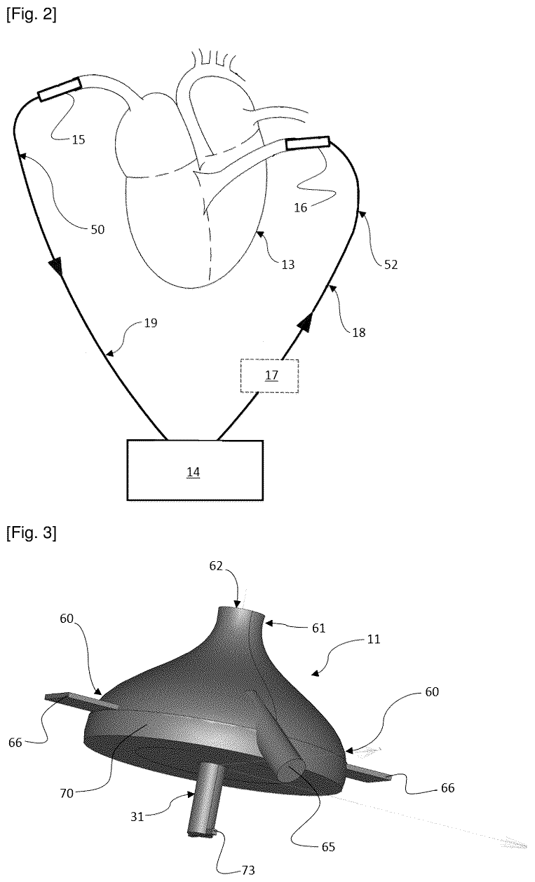

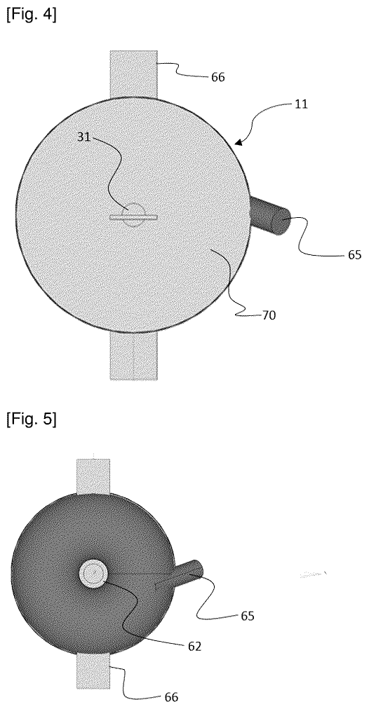

With reference to FIG. 1 or 2, the device according to the invention includes a pumping system 14 making it possible to partially or completely support the cardiac muscle by admitting a sufficient quantity of blood during the diastole phase of the cardiac cycle and reinjecting it during the systole phase of said cycle.

The operator introduces an admission cannula 15 (21 or 23 French or “Fr”, FRENCH representing ⅓ of a millimetre) capable of extracting the blood in the venous system of the patient 12 and an ejection cannula 16 capable (17 or 19 French) of injecting the blood into the arterial system of said patient 12. We will see hereinafter that a specific admission cannula 15 is advantageously used within the scope of the present invention. Nevertheless, the admission 15 and ejection 16 cannulas usually used on the extracorporeal circulation market (ECC, ECMO, ECLS) are also compatible with the invention.

The operator can perform:
left heart/left heart placement for partial assistance in the case of left heart failure, by positioning the admission cannula 15 at the right atrium, if the septum is perforated, the left atrium is drained, and the ejection cannula 16 at the abdominal aorta;
right heart/right heart placement for partial assistance in the case of right heart failure, by positioning the admission cannula 15 at a vena cava and the ejection cannula 16 at the pulmonary artery (FIG. 1 or 2);
right heart/left heart placement in the case of right heart and left heart failure, by positioning the admission cannula 15 at a vena cava and the ejection cannula 16 at the aorta. In the latter case, the lungs are also short-circuited and an oxygenation system 17 is placed in the derivation circuit to remove CO₂ of the blood and charge it with O₂ before reinjecting it into the body. This oxygenation system 17 is advantageously disposed after the reservoir 11, i.e. on the ejection portion 18 of the derivation circuit.

The cannulas 15, 16 may be placed percutaneously, in a cardiac and/or vascular catheterisation laboratory or in an intensive care unit or by a Mobile Circulatory Assistance and Emergency Medical Assistance Department Unit, by introducing them via a peripheral blood vessel and moving them near the heart 13, at the veins or arteries mentioned. They may also be placed surgically in an operating theatre, combined percutaneous puncture implantation and surgical opening of the vessels.

These cannulas 15, 16 are connected to the pumping system 14 via catheter type tubes, also compatible with those usually used for ECC to form, on one hand, the admission portion 19 and the ejection portion 18 of the derivation circuit.

Associated with this tube system of the portions 18, 19, a smart and independent purging system makes it possible to remove air, this system being known in the prior art. As we will see hereinafter, the reservoir has a shape and a disposition making it possible to remove bubbles, i.e. discharge them via the exit conduit 30 of the reservoir 11.

The pumping system 14 consists of a reservoir 11 making it possible to momentarily store a blood volume necessary for generating the blood flow and a piston 31 arranged with an actuator 10, of the linear motor type and any other equivalent means making it possible to transmit a forward/backward linear translation movement to said piston 31 so as to vary the volume of said reservoir 11 and pump the blood. Control means 40 are provided to automatically control the actuator 10. In the same way as for the admission cannula 15, a specific reservoir 11 and a specific membrane 41, actuated by the piston 31, will be described hereinafter because they are advantageously implemented in the device for temporary circulatory assistance or support of the heart 13 of a patient 12.

The control means 40, monitoring or controlling in particular the pumping system 14, are set up or implemented with a central unit 42 consisting of a computer, logic controller or similar.

Within the scope of the present application, the subject matter of the invention can be found in having a minimum set of pressure sensors 50, 51, 52 to anticipate any risk of collapse at the vena cava during the blood suction phase.

The pumping system 14 is controlled by the control means 40 and its central unit 42, in other words the blood flow pumped by this system is controlled by the central unit 42. The present invention thus provides having at least at least one pressure sensor 50 at the admission portion 19 of the derivation circuit, as close as possible to the vena cava. Obviously, this pressure sensor 50 is connected to the central unit 42/control means 40, i.e. the central unit 42 instantaneously receives the measurements obtained/originating from this pressure sensor 50.

Thus, if there is a pressure sensor 50 capable of being disposed in the admission cannula 15, such a sensor 50 is disposed therein. More probably, the pressure sensor 50 may be installed on or in the derivation circuit, between the admission cannula 15 and the reservoir 11.

Besides the location of placement of the pressure sensor 50, the important thing is that this pressure sensor 50 must have a measurement time constant of less than 20 milliseconds, advantageously less than 10 milliseconds, in order to detect the blood pressure and any modification thereof very rapidly and very regularly over time.

The central unit 42 has computing means wherein two types of alert relative to the pressure detected in the admission portion 19 of the derivation circuit of the blood of the patient 12 have been programmed. Firstly, an absolute pressure value threshold level which, if it is reached, triggers a modification of the pumping of the blood, conventionally by reducing the pumping flow rate. Then, the second alert consists of accelerating/decelerating the pressure, between two pressure measurements over time: once again, if this (acceleration/deceleration) threshold slope is reached, the suction of the blood is modified, conventionally by reducing or stopping it. Regarding the threshold slope, the predictive pressure progression program conventionally uses the Reynolds number of the Reynolds wave. The Reynolds number corresponds to a dimensionless number which is used in fluid mechanics. This quantity makes it possible to characterise a flow, in particular the nature of its state. It is thus possible to know whether a flow is laminar, transitory or turbulent.

At each heartbeat, an order of magnitude of the extracted blood volume is of the order of 50 ml (millilitre) but this can rise to 100 ml, depending on the patient. A natural cardiac output is between 2.5 and 4.5 l/min/m² (litres per minute per square metre). In other words, the larger the surface area of a human body (and therefore their weight), the greater the blood circulation. In practice, to adjust the weight or the volume of blood to be drawn in at each heartbeat, the physician considers the weight of the patient 12 and infers the volume therefrom at each heartbeat.

For a heart beating at 70 bpm (beats per minute), the suction time is approximately 0.56 seconds such that a normal output is approximately 90 ml/s (millilitres per second per systole) i.e. 5 l/min (litres per minute). However, in practice, for a pulsatile ECC/ECMO/ECLS, the physician aims for a mean output of 3 or 4 l/min. Thanks to the device according to the invention, it is possible to increase (or optionally reduce) the quantities of blood drawn in to correspond to actual cardiac function as closely as possible, without risking collapse at the vena cava.

The progression of venous pressures at the venae cavae and the atrium is very complex on account of very low pressures (2 to 4 millimetres of mercury on average) and substantial pressure variations in the proximal atrial zone. The atrial pressure profile (right atrium) follows a curve with 3 peaks per cycle—with one peak considered among these 3 peaks—corresponding to different events such as right heart contractions or tricuspid valve relaxation. The admission of blood into the device according to the invention is offset relative to the natural diastole by an offset of the order of approximately 0.25 seconds (i.e. between 0.2 and 0.3 seconds).

For example, for the pressure threshold value (first alert) and the pressure slope or acceleration threshold value (second alert):
if the pressure slope or acceleration measured is 50% less than what it should be during a suction cycle, then this means that there is an imminent risk of collapse, and the central unit immediately slows the suction flow rate;
if the absolute pressure falls below the threshold pressure value by 1 mm Hg (millimetre of mercury), then the central unit 42 immediately stops pumping for this suction cycle because the risk of collapse is very/too significant. Pumping restarts at the next suction cycle.

Obviously, these slope and absolute threshold values are variable according to the patients 12. Moreover, it should be noted here that the device according to the invention may function with a quantity of physiological saline solution—adapted to its mixture in or with the blood of the patient 12—optionally present in the reservoir 11, when suction is started in the derivation circuit. If this physiological saline solution initially present in the reservoir 11 is provided, the operating cycle of the device according to the invention starts with the introduction of this quantity or a part thereof into the body of the patient 12, which involves slightly increasing the overall pressure in the blood and therefore venous system of the patient 12. Whereas if the device starts drawing in the patient's blood, without the presence of the quantity or this volume of physiological saline solution in the reservoir 11, the patient's overall venous pressure will fall slightly on account of the suction of the blood.

Another important factor for these pressure slope and absolute pressure threshold values can be found in the determination of the blood viscosity of the patient 12. This is enabled by the presence of a second pressure sensor 51 present in the reservoir 11. Indeed, the dynamic load losses at the ends of a non-compliant resistive circuit comply with the following formula:

$$s . q = L [ ( Pi - Po ) - R . q ) ]$$

where Pi and Po are the pressures at the circuit entry and exit, L the system inertance, q and flow rate and s the Laplace variable.

In the device according to the invention, the flow rate is very accurately measurable thanks to the position of the piston 31 in the reservoir 11. The viscous friction resistance R.q proportional to the flow rate is then easily inferred. R is directly proportional to the viscosity (Poiseuille's law in laminar state or other laws after eddy establishment). It may also be noted that knowing the blood viscosity of the patient 12 makes it possible to determine the pressure at a location of the circuit, in this instance of the derivation circuit of the blood of the patient 12, in the knowledge of the pressure at another location of said circuit.

A third pressure sensor 52 is advantageously positioned in the ejection portion 18 of the derivation circuit so as to confirm or refine the calculated blood viscosity value of the patient 12. This sensor 52 is intended to read the pressure during the ejection phase, during the systolic wave.

This blood viscosity value of the patient 12 exerts a direct influence on the absolute pressure and slope threshold values to be defined to alert a risk of collapse at the vena cava, i.e. at the location where the patient's blood is extracted. It is easily understood that the higher the viscosity of the blood of a patient 12, independently of pressure, the higher the risk of collapse. In this case, the first and second alert values are modified/lowered to incorporate this feature in the risk of collapse at the venae cavae.

FIG. 3 illustrates a preferred embodiment of the reservoir 11. This reservoir 11 thus has a pear shape with a wide base 60 of diameter d1 substantially greater than the diameter d2 at its top 61, with a slight decrease in this diameter d1 from the base 60 on a first elevation portion of the reservoir 11 then a substantial/rapid decrease until the diameter d₂ is reached. The entry conduit 65, for the introduction of the drawn in blood, is oriented upward, i.e. in the direction of the top 61 of the reservoir 11, with an angle relative to this top 61 between 30° and 70°. Moreover, this entry conduit 65 enters obliquely into the reservoir 11, either with an angle relative to the surface of the reservoir between 10° and 80°, this entry conduit 65 not being perpendicular to the surface of the reservoir 11 and advantageously as tangential as possible, or according to the smallest possible angle, less than 40° or 30°. Such designs are adopted to prevent the risks of haemolysis and discharge air bubbles toward the top 61.

At the top 61 of the reservoir 11 the exit or ejection conduit 62 enters, with therefore its diameter d2. The reservoir 11 is disposed vertically, resting on its base 60 and its top 61 forms or determines the height of said reservoir 11. The reservoir 11 here has two tabs 66 for mechanically locking, by rotation in a locking slot 67, the reservoir 11 on a mount 68, seen in FIG. 6.

Finally, the reservoir 11 includes a membrane 70 attached under the base 60 of the reservoir 11, the top surface of this membrane 70 forming the bottom inner surface of the reservoir. This membrane 70 is made of a flexible or elastic material, elastomer or other, at least at its top face or surface, corresponding to the bottom inner surface of the reservoir 11. Advantageously, the membrane 70 is entirely made of the same flexible and elastic material, such as an elastomer. This membrane 70 houses a central insert, not seen in the appended figures, equipped with an actuation arm consisting of the piston 31, this piston 31 being attached to a hydraulic or pneumatic cylinder 10, capable of moving this insert linearly. The piston 31 is attached to a cylinder 10 thanks to a mechanical means 73 for engagement with, attachment or connection to said cylinder 10.

Advantageously, the central insert has a diameter representing between 40% and 60% of the diameter of the membrane 70, or of the top surface 71 of the membrane 70. It is noted here that the diameter of the membrane 70 or the top face/surface 71 of the membrane 70 is equal, or substantially equal, to the diameter d1 of the base of the reservoir 11.

Each step of movement of the cylinder 10 corresponds to in internal volume of the reservoir 11, this correspondence between the step of the cylinder 10 and the internal volume of the reservoir 11 being stored or saved in the control means 40. Relative to an initial position wherein the top face or surface of the membrane 41 is not moved by the central insert, corresponding to a maximum internal volume of the reservoir 11, the insert is moved under the action of the cylinder 10 such that the top surface of the membrane 70 is raised in the reservoir 11, hence reducing the internal volume of the reservoir 11.

The piston 31 and its central insert 72 can rise such that the top surface 71 of the membrane 70 to near the top 61 of the reservoir 11 so as to drain all of the latter 61.

Given the nature of the membrane 70 and the shape of the central insert 72, during the raising of the top surface of the membrane 70, the contours of the latter 70 follow the internal walls of the reservoir 11 tightly to any liquid.

According to another advantageous aspect of the invention, the admission cannula 15 measures approximately 70 centimetres (cm)—between 60 and 80 cm—and is introduced at the upper part of the leg, as can be seen schematically in FIG. 1: once introduced in full, it reaches the immediate vicinity of the heart 13.

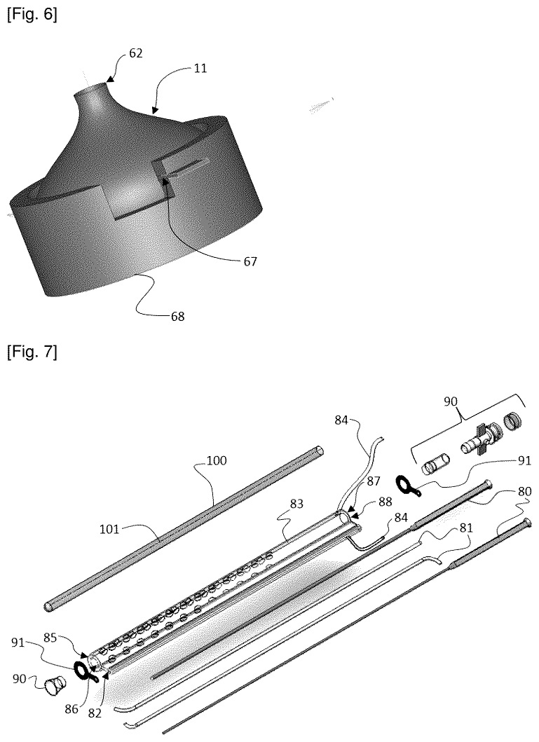

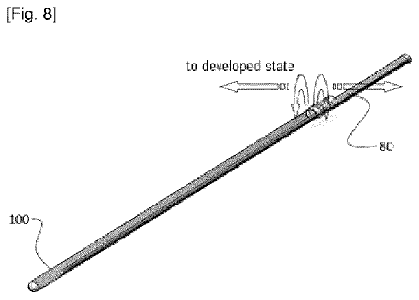

In FIG. 7, the different elements composing such an admission cannula 15 can be seen, some in duplicate or according to two alternative embodiments.

The admission cannula 15 includes at least one guiding mandrel 80 for pushing the cannula 15 for the introduction thereof 15 to its final position near the heart 13 of the patient 12, either of these mandrels 80 (according to the desired length) being used with the septum perforation and suction tools 81.

This admission cannula 15 also includes two septum perforation and suction tools 81, each having a slightly different shape from the other in order to facilitate the operator's work, on one hand, to perforate the septum then, on the other, to draw in the blood of the patient 12. This perforation of the septum is envisaged to discharge the blood overflow in the left ventricle, blood being likely to enter the lungs which are nearby and thus cause pulmonary oedema.

The guiding elements 80 as well as the perforation and suction tools 81 are intended to be introduced into a lateral duct 82 attached to the main body 83 of the admission cannula 15. This lateral duct 82 is attached to the main body 83 by plastic connection, using a metal connection insert or by any other suitable means.

A specificity of the admission cannula 15 according to the invention can be found in its ability to adopt two states: a first so-called retracted state wherein, on one hand, the lateral duct 82 is present contiguous to the main body 83 of the cannula 15 and, on the other, the main body 83, presented in the form of a hollow element, is not filled with fluid.

The main body 83 is indeed a hollow longitudinal body which can be filled with and emptied of, via the wired end conduits 84, a fluid, advantageously a liquid. The main body 83 includes at its distal end 85, near the heart 13 when disposed in the vena cava, a longitudinal suction opening 86 and at its opposite proximal end 87, an opening 88 to allow the drawn-in blood to enter the admission portion 19 of the derivation circuit to the reservoir 11.

The main body 83 of the admission cannula 15 also has the specificity of a plurality of lateral openings 89—of the order of several tens—on the circumference of this body 83 from the longitudinal suction opening 86.

Moreover, connection elements 90 can be seen in this FIG. 7 to be mounted on the proximal 87 and distal 85 ends of the main body 83. Two coupling elements 91 between the main body and the lateral duct may be observed, these coupling elements 91 making it possible to complete the connection or attachment between the main body 83 and the lateral duct 82.

As mentioned above, a major specificity of the invention can be found in the ability of the admission cannula 15 according to the invention to adopt two states, one retracted and the other developed. The retracted state of the main body 83 with the suction duct 82 is maintained using a mechanical means 100 capable of being destroyed or absorbed so as to automatically release the main body 83 and the lateral duct 82 to the developed state. In this instance, this mechanical means 100 consists of a temporary wrapper wherein the empty hollow main body 83 and the lateral duct 82 mounted against said body 83 are constrained when in the retracted position.

The temporary wrapper 100 includes a breakable longitudinal line 101. This breakable line 101 makes it possible to cut the wrapper 100 into two substantially equal parts and remove the wrapper 100 by pulling on a portion accessible to the operator, directly in that the proximal end 87 of the cannula 15 is not inserted into the body of the patient 12 or thanks to a protuberant part of the wrapper 100, not shown in the appended figures, outside said body 83 for removing said wrapper simply by pulling it. The breakable line 101 is activated readily starting with the proximal end 87 simply by pulling both parts of this temporary wrapper 100 or using a pull thread, not shown in the appended figures, to release the breakable line 101.

This breakable line 101 consists of a tear line pretreated in terms of thickness, nature of the material of the wrapper and pre-perforations of this line. This tear line 101 induces or allows a fragility enabling or allowing peeling of the temporary wrapper into two or more parts if there are a plurality of such breakable lines 101 on the wrapper 100. The temporary wrapper 100 is advantageously made of plastic.

Obviously, this breakable line 101 is a means to release or remove this temporary wrapper 100 while holding the retracted state of the admission cannula 15 but any other mechanical means making it possible to tear, cut and/or remove the temporary wrapper 100 so as to enable or allow automatically the developed state of the admission cannula 15 may be envisaged.

Although the invention has been described in connection with several particular embodiments, it is obvious that it is in no way limited thereto and that it comprises all of the technical equivalents of the described means as well as their combinations if these fall within the scope of the invention.

The arrangement of the different elements and/or means and/or steps of the invention, in the embodiment described above, should not be understood as requiring such an arrangement in all the implementations. In any case, it will be understood that various modifications may be made to these elements and/or means and/or steps, without deviating from the spirit and the scope of the invention. In particular:
the nature and the precise positioning of the first pressure sensor 50, as well as other advantageous and optional pressure sensors 51, 52;
the threshold value of the first (absolute value) and the second (slope) pressure alerts making it possible to trigger a modification of the blood suction.

The use of the verb “include”, “comprise” or “contain” and of its conjugated forms does not exclude the presence of other elements or other steps than those set out in a claim.

In the claims, any reference sign between parentheses should not be interpreted as a limitation of the claim.

1. Temporary circulatory assistance and support device for the heart (13) of a patient (12), the device including a derivation circuit of the blood reaching the heart (13) to move it to the output of the heart (13) of the patient (12), the derivation circuit using tubes interconnecting the following elements of said device:
at the admission portion (19) of the derivation circuit, an admission cannula (15) intended to be introduced into a vena cava of the patient (12) to extract blood from the venous system,
at the ejection portion (18) of the derivation circuit, an ejection cannula (16) intended to be introduced into a patient's aorta or pulmonary artery to inject blood into the arterial system of the patient (13),
a pumping system (14) including a reservoir (11) with variable internal volume, located between the admission portion (19) and the ejection portion (18), for the admission of the extracted blood followed by the ejection of this blood, said pumping system (14) being controlled by control means (40) such that the pumping of the blood, from its admission to its ejection, accounts in real time for the patient's physiological needs and the hemodynamic status of said patient (13), the pumping system (14) including at least a first pressure sensor (50), connected to the control means (40), disposed in the blood derivation circuit,
characterised in that the first pressure sensor (50) is disposed in the admission portion (19) and in that this pressure sensor (50) has a measurement time constant of less than 20 milliseconds so that the control means (40) modulate or stop the pumping of the extracted blood when the pressure increases/decreases above or below a threshold pressure acceleration/deceleration slope.

2. Circulatory assistance and support device according to claim 1, wherein the control means (40) also modulate or stop the pumping of the extracted blood when the pressure detected by the sensor (50) attains a threshold pressure value.

3. Circulatory assistance or support device according to claim 1, wherein the pressure sensor (50) has a measurement time constant of less than 10 milliseconds.

4. Circulatory assistance or support device according to claim 1 or 2, wherein the first pressure sensor (50) is located in or on the admission cannula (15), at a distance of at most 15 centimetres from the proximal end (87) of said cannula (15), or in the derivation circuit between the admission cannula (15) and the pumping system (14).

5. Circulatory assistance or support device according to any one of the preceding claims, wherein the pumping system (14) comprises a second pressure sensor (51) located in the reservoir (11) such that, thanks to the pressure readings of the first and the second sensor (50, 51), the control means (40) determine the viscosity of the extracted blood, this viscosity value being integrated or considered in said threshold pressure value and/or said threshold pressure acceleration slope.

6. Circulatory assistance or support device according to any one of the preceding claims, wherein the pumping system comprises a third pressure sensor (52) located in the ejection portion (18).

7. Circulatory assistance or support device according to any one of the preceding claims, wherein the pumping system (14) also includes a system for oxygenating the blood (17), preferably this system for oxygenating the blood (17) enabling blood oxygenation at the ejection portion (18).

8. Circulatory assistance or support device according to any one of the preceding claims, wherein the reservoir (11) has a pear shape with a base (60) of wide diameter d₁ reducing in its height to reach a top (61) of small diameter d₂ such that d₁ is at least equal to seven times d₂, i.e. d₁>7d₂, and in that the admission portion (19) enters the reservoir (11), via an entry conduit (65), oriented obliquely according to an angle between 10° and 60° relative to the surface of the reservoir (11) and upward—i.e. toward said top (61)—according to an angle between 10° and 50°.

9. Circulatory assistance or support device according to claim 7, wherein the reservoir (11) is disposed vertically, the base (60) extending along a horizontal plane, and the exit conduit (62) being located symmetrically, along a perpendicular and vertical plane relative to the extension plane of the base (60), to the top (61) of the reservoir (11).

10. Circulatory assistance or support device according to any one of the preceding claims, wherein the internal volume of the reservoir (11) is between 80 cm3 and 120 cm3, preferably between 95 cm³ and 105 cm³.
